(19) United States
(12) Patent Application Publication
(10) Pub. No.: US 2012/0217418 A1
(43) Pub. Date: Aug. 30, 2012

(54) WAVELENGTH CONVERSION OF EXCIMER-GENERATED UV LIGHT

(75) Inventors: Jeffrey I. Levatter, Solana Beach, CA (US); Michael R. Stewart, Doylestown, PA (US)

(73) Assignee: PhotoMedex, Montgomeryville, PA (US)

(21) Appl. No.: 13/277,955

(22) Filed: Oct. 20, 2011

Related U.S. Application Data
(63) Continuation of application No. 11/411,237, filed on Apr. 25, 2006, now abandoned.
(60) Provisional application No. 60/674,883, filed on Apr. 26, 2005.

(51) Int. Cl. G21K 5/00; A61N 5/067

(52) U.S. Cl. 250/461.1; 250/492.1

(57) ABSTRACT

An optical apparatus for treating tissue including an ultraviolet excimer laser configured to output ultraviolet light, an optical line configured to receive said ultraviolet light into a first end and to output said ultraviolet light from a second end, a wavelength converting device configured to receive said ultraviolet light output from said optical line and to produce longer wavelength emissions, and a delivery device configured to direct said longer wavelength emission to said tissue.

### CROSS-REFERENCE TO RELATED APPLICATIONS

This application is a continuation application of U.S. patent application Ser. No. 11/411,237, filed Apr. 25, 2006, also entitled “Wavelength Conversion of Excimer-Generated UV Light,” which claims priority under 35 U.S.C. §119(e) to U.S. Provisional Application Ser. No. 60/674,883, filed Apr. 26, 2005, entitled “Wavelength Conversion of Excimer-Generated UV Light,” which are both hereby incorporated herein by reference in their entirety.

### BACKGROUND

1. Field of the Invention

The present invention is directed to a method and apparatus for treating tissue with ultraviolet light.

2. Description of the Related Art

Ultraviolet (UV) light has many medical and surgical applications. In dermatology, 308 nanometer wavelength light obtained from an excimer laser has been demonstrated to have a therapeutic effect in the treatment of disease. Skin disorders such as psoriasis and vitiligo, for example, may be treated with relatively high dose exposure to such ultraviolet light. Precise delivery of therapeutic doses of energy, much like a titrated dose of medicine, may be employed. Accordingly, in certain treatments, a dose of light in a narrow, therapeutic wavelength range is used, and this dose is specifically directed onto an affected area of tissue with reduced exposure to adjacent healthy tissue. In such cases, an excimer laser operating at a narrow wavelength range at about 308 nanometers has found a role in the dermatologist's practice.

Some medically therapeutic practices, however, may have need for light sources that utilize other wavelengths to provide treatment. In a variety of cases, these other wavelengths need not necessarily be coherent or in phase when delivered to the tissue site. Indeed, it is not unusual to see in a single dermatologist's practice a 308 nm excimer laser alongside other non-laser, e.g., lamp-based, light sources that provide monochromatic or polychromatic, incoherent radiative therapy at wavelengths different from the wavelength provided by the laser. Among such light sources are the B-Clear™ manufactured by Lumenis and the Omnilux™ manufactured by PhotoTherapeutics Ltd.

### SUMMARY

In one aspect, the present invention provides an optical apparatus for treating tissue comprising an ultraviolet excimer laser configured to output ultraviolet light, an optical line configured to receive said ultraviolet light into a first end and to output said ultraviolet light from a second end, a wavelength converting device configured to receive said ultraviolet light output from said excimer laser and to produce longer wavelength emissions, and a delivery device configured to direct said longer wavelength emission to said tissue.

In another aspect, the present invention provides a method of treating a region of tissue, said method comprising generating ultraviolet light from an ultraviolet excimer laser, producing longer wavelength emission from said ultraviolet light, and illuminating said region of tissue with said longer wavelength emission.

In yet another aspect, the present invention provides an adapter comprising fluorescent material that emits longer wavelength light when illuminated with ultraviolet light from an excimer laser light source, said adapter configured to be attached to a handpiece.

In still another aspect, the present invention provides a kit comprising a plurality of adapters comprising fluorescent material, said adapters configured to emit different wavelength light when pumped by ultraviolet light from an excimer laser light source, said adapters configured to attach to a handpiece.

In yet still another aspect, the present invention provides a handpiece configured to receive a fluorescent material that emits longer wavelength light when illuminated with ultraviolet light from an excimer laser light source.

In a further aspect, the present invention provides an optical apparatus for treating tissue comprising an ultraviolet laser configured to output ultraviolet light, an optical line configured to receive said ultraviolet light into a first end and to output said ultraviolet light from a second end, a wavelength converting device configured to receive said ultraviolet light output from said ultraviolet laser and to produce longer wavelength emissions, and a delivery device configured to direct said longer wavelength emission to the tissue to be treated.

All of these embodiments are intended to be within the scope of the invention herein disclosed. These and other embodiments will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

These and other features, aspects, and advantages of the invention disclosed herein are described below with reference to the drawings of preferred embodiments, which are intended to illustrate and not to limit the invention. The drawings comprise four figures in which:

FIG. 1 schematically illustrates an example system that may be configured to emit excimer-generated UV light at one wavelength or to convert the UV light into longer wavelength light.

FIG. 2 schematically illustrates an example system that may be configured to emit laser light having a relatively short wavelength as well as incoherent light having a relatively long wavelength.

FIG. 3 schematically illustrates another example configuration of a wavelength conversion device for a UV laser.

FIG. 4 schematically illustrates a reflector at the proximal side of the medium to redirect and refocus backward-emitted light in a forward direction.

### DETAILED DESCRIPTION OF CERTAIN PREFERRED EMBODIMENTS

Although certain preferred embodiments and examples are disclosed below, it will be understood by those in the art that the invention extends beyond the specifically disclosed embodiments and/or uses of the invention and obvious modifications and equivalents thereof. Thus, it is intended that the scope of the invention herein disclosed should not be limited by the particular disclosed embodiments described below.

As described above, it is not unusual to see in a single dermatologist's practice a 308 nm excimer laser alongside other non-laser, e.g., lamp-based, light sources that provide monochromatic or polychromatic, incoherent radiative therapy at wavelengths different from the wavelength provided by the laser. Typically, multiple pieces of equipment mean multiple capital expenditures. As described herein, however, excimer UV light systems can be adapted to “step down” the emitted UV light—that is, they can produce light having a reduced optical frequency or longer wavelength. This longer wavelength light may be coherent. The longer wavelength light can also be incoherent to reproduce or mimic the effect of lamp-based devices.

Described herein are feasible systems and methods for safely and economically converting UV excimer light (i.e., light having a generally shorter wavelength) into light having a longer wavelength. With such systems and methods, a dermatologist can avoid multiple expenditures for multiple pieces of equipment. A single expenditure for an excimer-generated UV light source and some accessories may suffice, thereby reducing the cost of operation to the health care provider and the cost that is passed on to the patients.

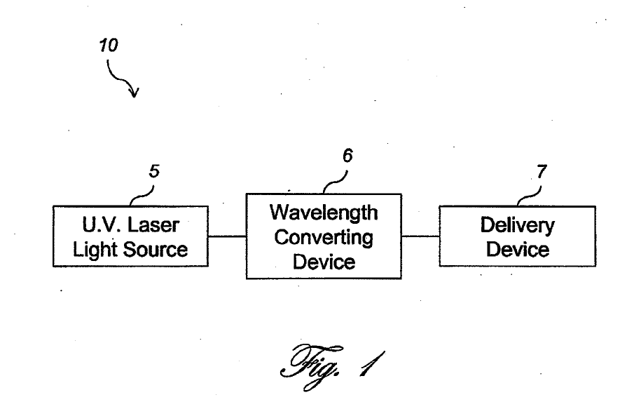

FIG. 1 schematically illustrates an example system 10 that may be configured to emit excimer-generated UV light at one wavelength, or the system 10 can be configured to convert the UV light into longer wavelength light. The system 10 comprises a UV laser light source 5, a wavelength converting device 6, and a delivery device 7. The UV laser 5 comprises an excimer laser. In various preferred embodiments, the laser 5 is a temporally coherent source (e.g., having a linewidth of about ±1 nanometer or less). The wavelength converting device 6 can be part of the delivery device 7 by fitting inside or temporarily fastening onto, e.g., the outside, of the delivery device 7. The wavelength converting device 6 may be removable. Although many configurations of such a system 10 are contemplated, two examples are further described below.

Because lasers may be more expensive than other types of UV light sources such as excimer lamps, using a UV laser light source in constructing a system for producing a long wavelength may not make a practical design. The cost of such a system may render the use of a laser a poor choice for the pump source. Practitioners, however, may already have such a laser light source specifically for other purposes for which lasers are particularly advantageous.

For example, lasers are generally spatially coherent and can produce a tightly focused beam for illuminating a small target region. Lasers also are generally temporally coherent and output a narrow range of wavelengths. Lasers therefore can provide a specific wavelength for medical treatment without also exposing the patient to additional wavelengths that are not as therapeutic and/or which may be harmful. For example, an excimer laser may provide a narrow wavelength band at about 308 that is therapeutically effective for treating certain dermatological conditions such as psoriasis, but which reduces exposure of the patient to other UV wavelengths that are not as therapeutically effective yet that increase the risk causing of skin cancer. The laser may also be focused tightly such that the diseased tissues and not the healthy tissue adjacent thereto is exposed to the UV light, again reducing the risk of cancer.

Recognizing that practitioners may already have excimer lasers for dermatological treatments, apparatus and methods are described herein that additionally provide for longer wavelengths of light when needed, even when such lasers would otherwise be impractical to include in a long wavelength light source. This recognition has yielded a variety of different designs that are both useful and are cost-effective for the practitioner.

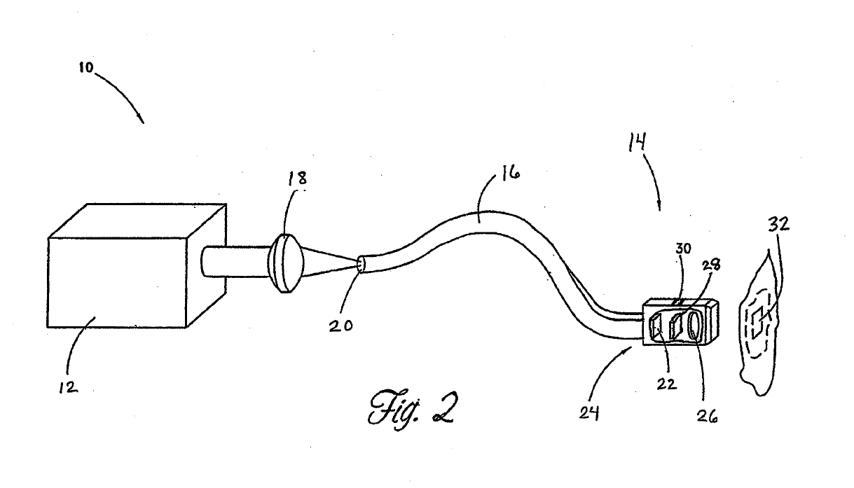

FIG. 2 schematically illustrates an example system 10 that may be configured to emit laser light having a relatively short wavelength, as well as incoherent light having a relatively long wavelength. The system 10 comprises an excimer laser 12 and a delivery device 14. This laser 12 may, for example, comprise a XeCl laser and output 308 nanometer laser light that may be employed in the treatment of dermatological disorders such as psoriasis and vitiligo, as well as other diseases. Example systems and methods for providing such treatment are described in U.S. patent application Ser. No. 10/799,337, entitled “Treatment of Skin Disorders with UV Light and Cooling,” which is incorporated herein by reference in its entirety. The systems and methods described in the incorporated application may apply as well to the systems and methods described in connection with FIG. 2.

The laser 12 may comprise an excimer laser such as, for example, that described in U.S. patent application Ser. No. 10/776,463 entitled “Rare Gas-Halogen Excimer Laser with Baffles” which is incorporated herein by reference in its entirety. Gas in the laser 12 includes excimers that may be used to generate laser energy, e.g., at a wavelength of about 308 nm for XeCl excimer lasers. The system 10 can be used with other lasers, including a large variety of other excimer lasers. These lasers may emit coherent light in a range of approximately 193 nanometers to approximately 351 nanometers.

With further reference to FIG. 2, the system 10 further comprises an optical line 16 such as a fiber optic cable or liquid light guide. A coupling lens 18 is disposed in an optical path between the laser 12 and the optical line 16, where the lens 18 couples light from the laser 12 into the optical line 16. The optical line 16 has an input end 20 and an output end 22. The input end 20 is proximal to the coupling lens 18 while the output end 22 is distal to the coupling lens 18.

The optical line 16 may be connected to a handpiece 24 that forms part of a delivery device 14. An example handpiece 24 is schematically shown in cutaway in FIG. 2. The handpiece 24 may include beamshaping optics 26 such as a lens that may collimate or focus light emitted from the output end 22 of the optical line 16. The beamshaping optics 26 may optionally be employed and may be implemented in a variety of ways to produce different optical outputs.

The system 10 further comprises a wavelength converting device 28 that may comprise a fluorescent material that fluoresces when illuminated by the light emitted from the laser 12. This fluorescent material may comprise a piece of plastic that is fluorescent. The fluorescent material may comprise organic material (such as a dye) or may comprise inorganic material (such as, e.g., cadmium sulfide). Other materials that may be pumped by the UV excimer radiation to produce light at longer wavelengths may also be employed.

In FIG. 2, the wavelength conversion device 28 is disposed in the delivery device 14 and more particularly in the handpiece 24. The handpiece 24 includes a slot 30 through which the wavelength converting device 28 may be inserted to be positioned to receive laser light emitted from the output end 22 of the optical line 16. Other arrangements may be employed to remove or interchange the wavelength conversion device 28.

During operation, the laser 12 emits coherent light of a relatively short wavelength, e.g., a wavelength in the range of approximately 193 nanometers to 351 nanometers. A preferred wavelength is 308 nanometers. The laser light is coupled into the optical fiber 16 at the proximal end 20 and is emitted from the distal end 22. This short wavelength optical energy is propagated through the wavelength converting device 28, which comprises a medium that will absorb the energy and then re-emit the energy at a longer wavelength. This longer wavelength may depend on the type of material. For example, the absorption and re-emission may result from the fluorescent properties of the material. In one embodiment wherein the laser 12 comprises an excimer laser that outputs 308 nanometer wavelength light that is propagated through a material comprising a fluorescent dye, the dye will absorb a portion of the UV laser energy and re-emit it at a longer wavelength characteristic of the particular dye.

The portion of the laser energy that is absorbed and converted may vary with the chemical composition of the material and with the length of the path along which the medium that the laser energy passes. The absorption of the light may, for example, depend exponentially on the distance traversed in the material.

Fluorescence depends upon a conversion of energy, and accordingly a fluorescent material will re-emit light at a wavelength longer—i.e., less energetic—than the wavelength that it has absorbed. If a dermatologist has an excimer laser at 308 nm in his or her practice, then the practice could theoretically command a plurality of wavelengths greater than 308 nm.

Table 1 lists examples of dyes, on the left, that can be pumped with excimer lasers to produce light in the corresponding range of wavelengths, on the right. Table 2 in lists other dyes. Other fluorescent materials and other wavelengths are also possible, and thus the wavelength converting device 28 is not limited to the dyes listed in Tables 1 and 2.

TABLE 1

| | WAVELENGTH | | WAVELENGTH |
|---|---|---|---|
| DYE | RANGE | DYE | RANGE |
| LDS-925/S-13 | 902-1023 | C-481 | 461-555 |
| LDS-867 | 878-960 | C-480 | 456-517 |
| IR-140 | 868-995 | LD-466 | 449-505 |
| IR-144 | 846-883 | C-460 | 438-482 |
| HITC | 812-909 | C-450 | 450-482 |
| LDS-821 | 785-900 | C-440 | 418-467 |
| LDS-765 | 770-833 | S-420 | 405-467 |
| Ox-750 | 719-807 | BisMSB | 411-431 |
| LDS-759 | 716-820 | POPOP | 411-466 |
| LD-700 | 693-797 | DPS | 393-419 |
| Ox-725 | 690-772 | E-428 | 408-439 |
| LDS-720 | 680-775 | E-417 | 398-430 |
| Ox-720 | 653-728 | E-416 | 404-427 |
| NB-690 | 656-740 | E-411 | 392-421 |
| CV-670 | 634-703 | E-404 | 392-415 |
| DCM | 602-713 | E-398 | 387-419 |
| SR-640 | 612-670 | E-392A | 373-397 |
| R-640 | 608-672 | E-389 | 375-407 |
| KR-620 | 597-646 | E-384 | 371-397 |
| R-610 | 594-644 | E-376 | 362-388 |
| R-590 | 553-615 | E-360 | 342-371 |
| R-560 | 542-578 | E-351 | 339-360 |
| F-555 | 518-596 | PBBO | 378-420 |
| F-548 | 540-587 | QUI | 368-402 |
| C-540A | 513-612 | TBS | 365-410 |
| C-525 | 508-550 | BBQ | 359-405 |
| C-510 | 495-538 | PBD | 350-386 |
| C-503 | 476-562 | BPBD | 353-386 |
| C-500 | 459-564 | DMQ | 346-377 |
| C-498 | 485-525 | TMQ | 344-366 |
| LD-489 | 471-535 | PTP | 322-358 |
| C-487 | 470-524 | DMT | 311-353 |

TABLE 2

| | | | | | |
|---|---|---|---|---|---|
| Q-Switch 5 | R-610 | PPO | C-522 | LD-690 | LDS-821 |
| IR-26 | R-590 | BPBD | C-521 | CV-670 | DOTC |
| DNTPC | P-597 | TMQ | C-510 | R-640 | Ox-750 |
| DNDTPC | P-580 | PTP | C-504 | KR-620 | LD-800 |
| IR-140 | P-567 | LDS-867 | C-503 | DCM | LD-700 |
| LDS-925/S-13 | R-575 | DTTC | LD-490 | R-610 | LD-690 |
| IR-125 | R-560 | IR-140 | LD-489 | R-590 | LDS-751 |
| IR-144 | F-548 | LDS-821 | C-498 | F-548 | Ox-725 |
| LDS-867 | DSF | HITC | C-487 | C-540A | LDS-698 |
| DTTC | C-540A | LD800 | LD-466 | C-503 | LD-688 |
| LDS-860 | C-522 | DOTC | C-480 | C-500 | SR-640 |
| HITC | C-500 | Ox-750 | LD-473 | C-481 | DCM |
| LDS-821 | LD-490 | Ox-725 | C-460 | C-480 | R-640 |
| LDS-798 | LD-489 | LD-700 | C-450 | LD-473 | KR-620 |
| DOTC | C-481 | NB-690 | C-445 | LD-466 | R-610 |
| LDS-765 | C-480 | Ox-720 | C-440 | C-460 | R-590 |
| LDS-759 | LD-473 | SR-640 | LD-425 | C-450 | R-560 |
| LDS-751 | LD-466 | CV-670 | LD-423 | C-440 | P-567 |
| LD-700 | C-460 | R-640 | LD-390 | LD-425 | DSF |
| Ox-750 | C-450 | R-610 | BBQ | BisMSB | P-580 |
| LDS-750 | C-445 | KR-620 | BPBD | POPOP | P-556 |
| Ox-725 | C-440 | R-590 | PTP | S-420 | C-540A |
| LDS-730 | S-420 | R-575 | IR-140 | DPS | C-540 |
| LDS-722 | BisMSB | R-560 | IR-125 | PBBO | C-535 |
| LDS-720 | E-428 | F-555 | HITC | α NPO | C-515 |
| NB-690 | E-417 | P-605 | HIDC | BBQ | C-510 |
| LD-688 | E-416 | P-597 | LDS-821 | E-384 | C-498 |
| LDS-698 | E-411 | P-580 | DOTC | PQP | C-487 |
| DODC | E-404 | P-567 | Ox-750 | PPO | C-480 |
| CV-670 | E-398 | P-556 | Ox-725 | PBD | C-460 |
| Ox-720 | E-392A | P-546 | LDS-722 | BPBD | C-450 |
| DCM | E-389 | C-545 | LDS-698 | S-14 | S-420 |
| SR-640 | E-384 | C-540A | LD-700 | LDS-925/S-13 | E-389 |
| R-640 | E-376 | C-540 | NB-690 | IR-140 | E-400E |
| P-650 | LD-390 | C-525 | LD-688 | HITC | E-392E |
| KR-620 | BBQ | C-523 | Ox-720 | LDS-860 | E-377E |

More complex effects may be achieved by combining different materials that re-radiate light at different wavelengths. For example, mixing dyes or layering media having different dyes can produce a plurality of wavelength outputs. Treatments involving a number of different wavelengths may thereby be realized.

The direction of light emanating from the wavelength converting device 28 may be altered by the beamshaping optics 26. For example, the re-emitted light may be focused or collimated. The laser energy is then delivered to a treatment site 32 such as that shown in FIG. 2.

In various preferred embodiments, the wavelength converting device 28 is interchangeable. The wavelength converting device 28 can be removed and replaced with another wavelength converting device that outputs another wavelength. A variety of different treatments at different wavelengths can therefore be accommodated in this manner. The wavelength converting device 28 may also be removed to permit the 308 nanometer coherent laser light to irradiate the treatment area 32. Introduction and removal of the wavelength converting device 28 may be through the slot 30 in the handpiece 24, as shown in FIG. 2. Other configurations are possible.

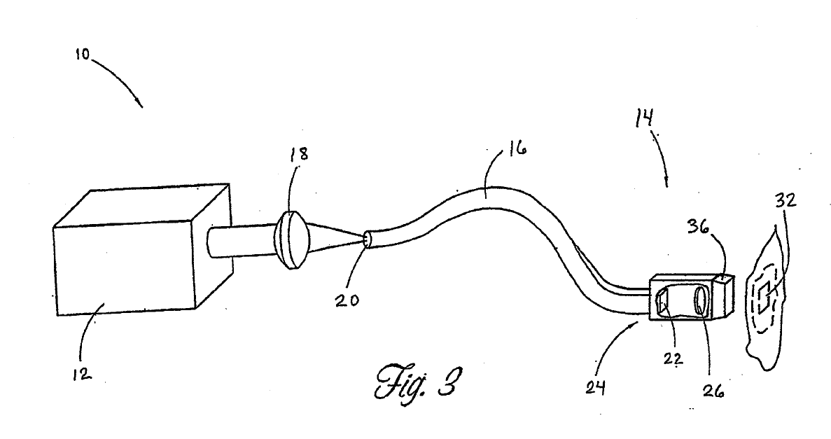

Another example configuration of a wavelength converting device is illustrated in FIG. 3. This device also includes a UV excimer laser 12. The embodiment of FIG. 3 also shows a removable end piece 36 that can perform the same optical function as the wavelength converting device 28 illustrated in FIG. 2. The end piece 36 can comprise a fluorescent material, such as fluorescent plastic. The removable end piece 36 can attach to the delivery device 14 using threads, a snap, etc. The removable end piece 36 can be connected to the delivery device 14 using arrangements other than those specifically recited herein as well as other methods, both those well known and those yet to be devised. The removable end piece 36 can be one of a large set of available end pieces that can be attached and detached depending on the procedure. The end pieces may be reusable or disposable.

The fluorescent material may be at the distal end of the hand piece 24 such that collector or focusing optics are not needed to direct the fluorescent radiation to the patient. The end piece 36 may be secured to the handpiece 24 and the handpiece 24 may be brought proximate to the area 32 to be treated for a particular procedure. The fluorescent material may be contacted to the skin or may be a small distance from the tissue in some cases.

Still other variations are possible. For example, the fluorescent material may be included elsewhere. The fluorescent material need not be included in or attached to the handpiece 24. In certain embodiments, for example, the fluorescent material may be disposed in the optical path between the laser 12 and the optical line 16 (e.g., fiber line or liquid light guide). Other configurations are also possible.

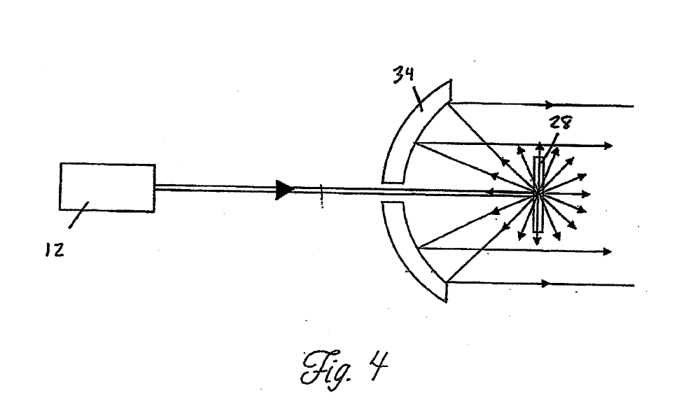

Referring to FIG. 4, in certain embodiments, light from the wavelength converting device 28 may be radiating in all directions. A reflector 34 at the proximal side of the medium 28 may be employed to redirect and refocus backward-emitted light in a forward direction, as shown in FIG. 4. Efficiency may thereby be improved. Other variations are also possible.

As described above, lasers may generally not be a practical choice to pump a fluorescent material to generate long wavelength spatially and temporally incoherent radiation. Lasers, which are generally spatially and temporally coherent, are typically more complex and costly than alternative spatially and temporally incoherent sources such as lamps. However, systems and methods for producing longer wavelength incoherent light from coherent UV lasers may be advantageously used when a practitioner already has a UV laser. This recognition has led to a variety of embodiments which are disclosed herein. In various embodiments, for example, additional output wavelengths can be provided by, for example, a 308 nm excimer laser, by adding a relatively low cost and simple fluorescent converter that transforms the 308 nm UV source to almost any longer wavelength. The conversion is non-coherent and the resultant light is also incoherent, but can be intense and therefore useful for a variety of medical dermatological applications. Such a system may allow the creation of additional uses and possible new medical therapies.

Use of a coherent laser to produce longer wavelength incoherent light by pumping fluorescent material can provide advantages over optical pumps comprising incoherent light sources. For example, lamps, being temporally incoherent, produce wide wavelength band. Similarly, pumping a fluorescent material with a lamp may also yield a wide wavelength distribution and produce wavelengths other than the desired wavelengths. In contrast, a coherent laser generally produces a narrow band, which can also result in a narrower wavelength distribution from the fluorescent material. Additionally, as described above, different fluorescent materials may provide different wavelengths. Pumping a variety of different types of fluorescent material with narrow band laser light therefore can provide increased control over the selection of the wavelength of the incoherent light.

Devices utilizing lamps and other incoherent sources may also result in poor efficiency of conversion. Lamps, for example, may require a collector and/or an amplifier to direct a sufficient amount of light energy from the lamp to the target. Laser beams may be easier to couple into an optical line, e.g., optical fiber or liquid light guide, than a beam from an incoherent source such as a lamp which may require more complex coupling optics. Lasers, which produce a more intense output from the fluorescent material also will require less time for the treatment as more energy is directed to the target region. Although the length of the treatment may not be noticeably affected in many circumstances, the more intense light may activate different processes that a less intense beam would not. For example, in treating vitiligo, faster, more intense delivery of energy to the melanocytes may yield more efficient re-pigmentation of the skin.

As described above, the UV light may be used to treat psoriasis, vitiligo, and other diseases. The longer wavelength light may be used to treat cancer or acne, to remove wrinkles or tattoos, or may be used for other applications, as well.

While the foregoing detailed description discloses several embodiments of the present invention, it should be understood that this disclosure is illustrative only and is not limiting of the present invention. It should be appreciated that the specific configurations and operations disclosed can differ from those described above, and that the structures and methods described herein can be used in other contexts.

What is claimed is:

1. An optical apparatus for treating tissue comprising:
an ultraviolet excimer laser configured to output ultraviolet light;
an optical line configured to receive said ultraviolet light into a first end and to output said ultraviolet light from a second end;
a wavelength converting device configured to receive said ultraviolet light output from said excimer laser and to produce longer wavelength emissions; and
a delivery device configured to direct said longer wavelength emission to said tissue.

2. The optical apparatus of claim 1, wherein said excimer laser comprises a XeCl laser.

3. The optical apparatus of claim 1, wherein said excimer laser is configured to emit light having a wavelength of between about 300 and 310 nanometers.

4. The optical apparatus of claim 1, wherein said optical line comprises a flexible light guide.

5. The optical apparatus of claim 1, wherein said optical line comprises a fiber optic or a liquid light guide.

6. The optical apparatus of claim 1, wherein said optical line comprises a fiber optic bundle.

7. The optical apparatus of claim 1, wherein said wavelength converting device comprises fluorescent material.

8. The optical apparatus of claim 7, wherein said fluorescent material comprises organic material.

9. The optical apparatus of claim 7, wherein said fluorescent material comprises inorganic material.

10. The optical apparatus of claim 1, wherein said wavelength converting device is disposed within said delivery device.

11. The optical apparatus of claim 1, wherein said wavelength converting device is attached to the outside of said delivery device.

12. The optical apparatus of claim 11, wherein said wavelength converting device is attached to a distal end of said delivery device.

13. The optical apparatus of claim 1, wherein the wavelength converting device is configured to be removable.

14. The optical apparatus of claim 1, wherein said delivery device comprises a handpiece optically connected to said optical line.

15. The optical apparatus of claim 14, wherein said wavelength converting device is included on a detachable adapter for connection to said handpiece.

16. The optical apparatus of claim 14, wherein said handpiece includes an opening for insertion of said wavelength converting device.

17. The optical apparatus of claim 1, wherein said wavelength converting device is disposed in an optical path between said excimer laser and said optical line.

18. A method of treating a region of tissue, said method comprising:
generating ultraviolet light from an ultraviolet excimer laser;
producing longer wavelength emission from said ultraviolet light; and
illuminating said region of tissue with said longer wavelength emission.

19. The method of claim 18, further comprising propagating said ultraviolet light through an optical line.

20. The method of claim 19, further comprising coupling said ultraviolet light from said optical line to a delivery device.

21. The method of claim 20, further comprising positioning said delivery device with respect to said region of tissue to illuminate said region of tissue with said longer wavelength emission.

22. The method of claim 21, further comprising moving said delivery device by hand to illuminate different portions of said tissue with said longer wavelength emission.

23. The method of claim 22, further comprising including a wavelength converting device in or on said delivery device to produce said longer wavelength emission.

24. The method of claim 25, further comprising removing said wavelength converting device from said delivery device and delivering ultraviolet light with said delivery device.

25. An adapter comprising fluorescent material that emits longer wavelength light when illuminated with ultraviolet light from an excimer laser light source, said adapter configured to be attached to a handpiece.

26. The adapter of claim 27, further comprising a connector portion that mates with said handpiece.

27. The adapter of claim 27, wherein said connector portion comprises threading, a snap-fit, at least one magnet, at least one screw, or at least one latch.

28. The adapter of claim 27, wherein said fluorescent material comprises fluorescent plastic.

29. A kit comprising a plurality of adapters comprising fluorescent material, said adapters configured to emit different wavelength light when pumped by ultraviolet light from an excimer laser light source, said adapters configured to attach to a handpiece.

30. The kit of claim 29, wherein said adapters comprise connector portions that mate with said handpiece.

31. The kit of claim 30, wherein said connector portions comprise threading, a snap-fit, at least one magnet, at least one screw, or at least one latch.

32. A handpiece configured to receive a fluorescent material that emits longer wavelength light when illuminated with ultraviolet light from an excimer laser light source.

33. The handpiece of claim 32, wherein said handpiece includes a holder therein configured to hold said fluorescent material in said handpiece so as to receive said ultraviolet light.

34. The handpiece of claim 32, wherein said handpiece includes a holder thereon configured to hold said fluorescent material to the outside of said handpiece.

35. The handpiece of claim 32, further comprising a lens in said handpiece for receiving said longer wavelength light.

36. An optical apparatus for treating tissue comprising:
an ultraviolet laser configured to output ultraviolet light;
an optical line configured to receive said ultraviolet light into a first end and to output said ultraviolet light from a second end;
a wavelength converting device configured to receive said ultraviolet light output from said ultraviolet laser and to produce longer wavelength emissions; and
a delivery device configured to direct said longer wavelength emission to the tissue to be treated.
